(19) United States
(12) Patent Application Publication
(10) Pub. No.: US 2019/0336315 A1
(43) Pub. Date: Nov. 7, 2019

(54) SOFT DYNAMIC ANKLE-FOOT ORTHOSIS EXOSUIT FOR GAIT ASSISTANCE WITH FOOT DROP

(71) Applicants: Panagiotis Polygerinos, Gilbert, AZ (US); Joshua Hsu, Flagstaff, AZ (US); Skyler Moore, Holbrook, AZ (US); Carly Thalman, Mesa, AZ (US)

(72) Inventors: Panagiotis Polygerinos, Gilbert, AZ (US); Joshua Hsu, Flagstaff, AZ (US); Skyler Moore, Holbrook, AZ (US); Carly Thalman, Mesa, AZ (US)

(21) Appl. No.: 16/396,409

(22) Filed: Apr. 26, 2019

Related U.S. Application Data
(60) Provisional application No. 62/663,910, filed on Apr. 27, 2018.

(51) Int. Cl. A61F 5/01; A61H 3/00

(52) U.S. Cl. CPC A61F 5/0113; A61H 3/00; A61H 2201/165; A61H 2003/007; A61F 2005/0155

(57) ABSTRACT

A soft robotic ankle-foot orthosis exosuit includes a brace configured to be worn on a user's foot, a first soft actuator, a second soft actuator, and a pneumatic system. The first soft actuator is coupled to the brace so that it is configured to be positioned proximate a top of the user's foot. The second soft actuator is also coupled to the brace and is configured to be positioned proximate a side of the user's foot. The pneumatic system is configured to change an internal pressure of the first soft actuator and the second soft actuator.

### CROSS-REFERENCE TO RELATED APPLICATIONS

This application claims priority to U.S. Application No. 62/663,910, filed Apr. 27, 2018, the entire contents of which are incorporated herein by reference.

### BACKGROUND OF THE DISCLOSURE

Ankle and Foot Orthoses (AFOs) are biomedical devices designed to target and guide the movement of the foot. Dorsiflexion is the movement that brings the toes closer to the body. Ankle and foot dorsiflexors consists of the tiabilis anterior; the extensor, halluces longus; and the extensor, digitrum longus. All three muscles assist the ankle in properly orienting the foot during walking by ensuring the foot clears the ground in the swing phase of the gait cycle, and firmly plants during the stance phase.

Damage to these muscles or to the nerves surrounding them can lead to severely impaired walking if not properly treated. If these three muscles are weakened by nerve injury, muscular atrophy, or disease, equinovarus deformity can result. Multiple Sclerosis (MS) is one of a variety of neural muscular diseases that affect the muscular movement in the lower extremities and require the use of an AFO. Impaired walking is also known as steppage gait, or “foot drop.” Affected individuals cannot complete the full range of dorsiflexion in the foot while walking, and often compensate by swinging the hip and knee in an exaggerated fashion in an attempt to keep the foot from catching on the ground during the swing phase of the gait cycle. These overcorrections made by the patient results in an abnormal gait, which can then later lead to discomfort. Foot drop can cause an individual a severe amount of pain over time. This may include back, hip and knee problems, as well as an increased probability of falling, tripping or damaging the foot even further during walking. This condition is typically treated through physical therapy, and providing the user with a rigid brace to prevent the foot from dropping as gait training is performed.

Existing devices to assist sufferers of foot drop aim to brace the ankle at the standard 90-degree angle during the stance phase. This prevents the ankle from buckling when a load is applied, and holds the foot up to prevent drag during the swing phase of the gait cycle. However, these devices can be uncomfortable and only supply rigid support rather than providing dynamic assistance to the user throughout the cycle in order to promote muscle growth.

Many of these braces are rigid and hold the ankle joint firmly in place while the user is wearing the device, and do not allow for movement at the ankle joint. While many similar types of solutions exist on the market today, many of these solutions do not address the disturbance to the gait cycle as the user wears the device. These braces hold the ankle in place but overlook the importance of allowing the user to move his or her ankle naturally as he or she walks.

### SUMMARY OF THE DISCLOSURE

The easily conformable design associated with soft robotics performs well when paired with the complex biomechanics of the body. Soft robotics have been proven in the past to be a successful approach to rehabilitative devices when used in the context of soft exosuits, specifically for the lower body.

Soft robotics can be utilized to provide the active support necessary to correct the user's foot drop while allowing the user to strengthen ability to lift the foot which will ideally lead to a greater recovery than only the use of a static orthosis. In addition to the added support, this orthosis may also be significantly more comfortable to wear than its rigid counterparts.

The disclosure herein describes a soft robotic ankle-foot orthosis (AFO) exosuit for the purpose of correcting ankle orientation to aide in the restoration of a natural gait cycle in individuals suffering from foot drop. The exosuit may include a soft, pneumatically controlled actuator made from a thermally-bonded material encased in an inextensible fabric, and may be controlled off of a portable pump and on-board logic controller. The soft AFO exosuit uses force sensitive resistive (FSR) sensors embedded in custom insoles to detect what stage of the gait cycle the user is in, as well as IMU sensors to ensure the angle of the foot is sufficient to clear the floor.

The AFO's individual components were tested to ensure that they would provide the necessary forces needed for foot stability and for lifting the foot during the swing phase. The system as a whole was tested both on a designed test platform and on a test participant. The AFO was able to supply the needed forces when the user walked at a slow pace such as for a patient recovering from an injury.

In one embodiment, a soft robotic ankle-foot orthosis exosuit includes a brace to be worn on a user's foot, a first soft actuator, a second soft actuator, and a pneumatic system. The first soft actuator is coupled to the brace so that it is positioned proximate a top of the user's foot. The second soft actuator is also coupled to the brace and is to be positioned proximate a side of the user's foot. The pneumatic system changes an internal pressure of the first soft actuator and the second soft actuator.

In another embodiment, a soft robotic ankle-foot orthosis exosuit includes a brace to be worn on a user's foot, sensors coupled to the brace, a first soft actuator, a second soft actuator, and a pump. The first soft actuator is coupled to the brace and provides dorsiflexion assistance to the user's foot. The second soft actuator is coupled to the brace and limits inversion or eversion of the user's foot. The pump is in fluid communication with the first soft actuator and the second soft actuator. The pump is adjusts an internal pressure of the first soft actuator and an internal pressure of the second soft actuator based on measurements from the sensors.

In yet another embodiment, a soft robotic ankle-foot orthosis exosuit includes a brace to be worn on a user's foot, an actuator array, a first side actuator, a second side actuator, and a pneumatic system. The actuator array is coupled to the brace proximate a top of the user's foot. The actuator array includes multiple interconnected fluid bladders. The first side actuator is coupled to a first side of the brace, and the second side actuator is coupled to a second side of the brace. The pneumatic system is supported by the user away from the brace. The pneumatic system substantially uniformly changes an internal pressure in the interconnected fluid bladders and substantially uniformly changes an internal pressure in the first and second side actuators.

Other aspects of the disclosure will become apparent by consideration of the detailed description and accompanying drawings.

### BRIEF DESCRIPTION OF THE DRAWINGS

FIG. 1 is a concept illustration of a soft AFO exosuit design and implementation according to an embodiment of the present disclosure.

FIG. 2 is an exploded view of the AFO of FIG. 1, highlighting actuators and sensors.

FIG. 3 is an exploded view of a soft AFO exosuit design and implementation according to another embodiment of the present disclosure.

FIGS. 4A-4C are illustrations of the AFO of FIG. 3, highlighting a put on/removal process.

FIG. 5 is a block diagram detailing a basic logic loop of the exosuit of FIG. 1 or FIG. 3.

FIG. 6A is a free body diagram of an actuator array of the soft AFO exosuit of either FIG. 1 or FIG. 3 during a swing phase assist, and is used to calculate a size and geometry of the actuator array.

FIG. 6B illustrates a free body diagram of the side actuators of the soft AFO exosuit of either FIG. 1 or FIG. 3 during a standing condition to calculate a size and geometry of the side actuators.

FIG. 7A illustrates diagrams used to calculate specific sizes and shapes of the actuator array.

FIG. 7B illustrates diagrams used to calculate specific sizes and shapes of the side actuators.

FIG. 8A is a free body diagram of the actuator array and approximate measurements of the foot.

FIG. 8B is a free body diagram of the actuator array post inflation assuming each unit (n) forms a perfect circle when inflated.

FIG. 8C is a deflated length of a single unit of the actuator array.

FIG. 9A is a deflated state of a single two-unit actuator array.

FIG. 9B is the two-unit actuator array of FIG. 9B, when inflated.

FIG. 9C is a side view of the inflated actuator array of FIG. 9B.

FIG. 10A shows a contraction percentage of the actuator array varying the height of each unit as a ratio of the width.

FIG. 10B shows a contraction percentage of the actuator array varying the side gaps of each unit as a ratio of the width.

FIG. 11A illustrates results of torque testing on the actuator array.

FIG. 11B illustrates a setup of torque testing on the actuator array.

FIG. 12A represents the results of testing the different sized bladders.

FIG. 12B is a demonstration of testing the stiffness of the different sized actuators.

FIG. 13A illustrates the setup and results of a quasistatic isometric test for the actuator array.

FIG. 13B illustrates the setup and results of a constant pressure test for the actuator array.

FIG. 14A is a constant pressure test performed with various applied loads at the tip of a cantilever beam.

FIG. 14B is a constant load test with incrementally increased pressure values.

FIG. 15 is a diagram detailing the test platform used to measure range of motion.

FIG. 16A is a side view of the test platform showing resistive cabling.

FIG. 16B represents the results measured from testing the front bladder.

FIG. 17 illustrates data collected from the sensors on the AFO over the course of one gait cycle.

FIG. 18A is a graph depicting the state of the system during a single gait cycle from heel strike to heel strike showing the output of the fluidic pressure sensors indicating when each soft actuator is active.

FIG. 18B is a graph depicting the state of the system during a single gait cycle from heel strike to heel strike showing the timing of the ground reaction force (GRF) readings indicating which part of the foot is on the ground and if it has crossed the threshold to trigger activation/deactivation of corresponding actuators.

FIG. 18C is a graph depicting the state of the system during a single gait cycle from heel strike to heel strike showing maximum voluntary contraction surface electromyography (sEMG) data of the tibialis anterior muscle with sEMG placement illustrated just below the knee.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. Use of “including” and “comprising” and variations thereof as used herein is meant to encompass the items listed thereafter and equivalents thereof as well as additional items. Use of “consisting of” and variations thereof as used herein is meant to encompass only the items listed thereafter and equivalents thereof. Unless specified or limited otherwise, the terms “mounted,” “connected,” “supported,” and “coupled” and variations thereof are used broadly and encompass both direct and indirect mountings, connections, supports, and couplings.

In general, the present disclosure relates to a device that allows the user to regain control of the dorsiflexion in the user's foot while walking, all while avoiding interference with the user's natural gait cycle to prevent further injuries to the body during rehabilitation of the ankle.

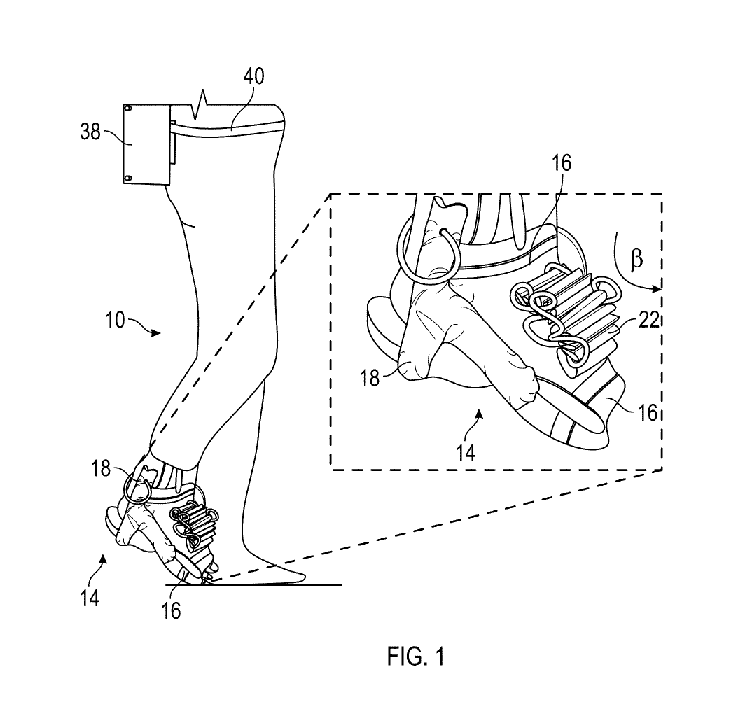

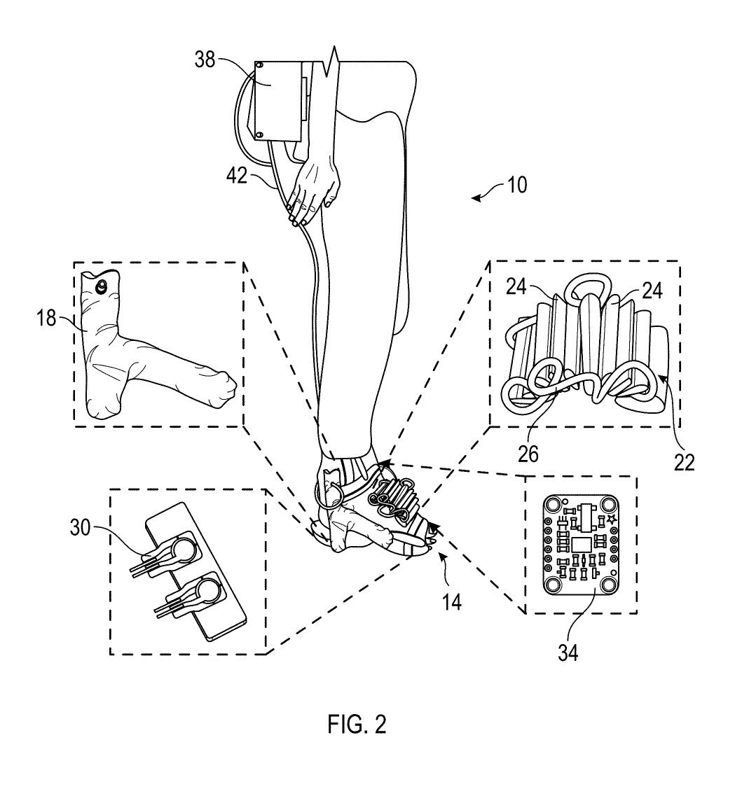

As shown in FIGS. 1 and 2, an exosuit 10 (e.g., a soft Ankle and Foot Orthoses (AFO) exosuit) includes a brace or sock 14 worn on a foot of the user. The sock 14 may be made from an elastic spandex material, which may stretch to assist a user in putting on or taking off the sock 14. The sock 14 may also be adjustable to fit the user by adjusting securement members 16 (e.g., Velcro straps) at the top and bottom of the exosuit 10. The sock 14 may be custom made for each individual user in order to provide a snug fit.

Side actuators 18 are placed on either side of the ankle, across the joint at the talus. Each side actuator 18 branches off to reach the bottom of the calcaneus and then down the inner and outer edges of the foot to reach the first and fifth metatarsals, respectively (i.e., the side actuator 18 splits to simultaneously reach the front and rear of the foot). The side actuators 18 act as an ankle wrap brace and inflate in order to provide support and stability to the patient. The side actuators 18 limit inversion or eversion caused by supination or pronation of the ankle (e.g., which may be seen in cases of ankle instability). In the illustrated embodiment, each of the side actuators 18 is made from a single fluid bladder.

A bending actuator or actuator array 22 is placed on the front of the sock 14 between the side actuators 18. As shown in FIG. 2, the actuator array 22 is made from a series of interconnected bladders 24. A fluid conduit 26 (e.g., plastic tubing) extends between each of the bladders 24 to inflate or deflate each of the bladders 24. The actuator array 22 creates a bending motion B (FIG. 1) when inflated. This allows the exosuit 10 to provide dorsiflexion assistance during the swing phase of the gait cycle to ensure that the foot does not catch on the ground.

In the illustrated embodiment, each actuator 18, 22 is made from a Thermoplastic Polyurethane (TPU) material which is thermally bonded to create an airtight seal. This forms an air bladder (e.g., bladder 24), which is then encased in an inextensible fabric (e.g., 40D Ripstop Nylon) of the same net shape as the TPU air bladder. The TPU material has a low tensile strength and cannot withstand being inflated over a maximum pressure (e.g., 40 kPa). Encasing the bladder 24 in an inextensible fabric creates a single actuator 18, 22 that may be inflated up to the limits of the inextensible fabric. As a result, each actuator 18, 22 can be over pressurized to create a rigid structure.

The exosuit 10 also includes force sensors 30 (e.g., force sensitive resistive (FSR) sensors) in order to inflate and exhaust the actuators 18, 22 at the correct times and pressures. In the illustrated embodiment, four force sensors 30 are coupled to the bottom of the sock 14 to detect the placement of the foot. Two force sensors 30 are disposed below the ball of the foot and two force sensors 30 are disposed below the heel. The placement of the force sensors 30 allows the exosuit 10 to determine where the user's weight is distributed.

The exosuit 10 also includes Inertial Measurement Units 34 (IMUs). In the illustrated embodiment, one IMU 34 is coupled to the exosuit 10 proximate the top of the foot, and one IMU 34 is coupled to the exosuit 10 proximate the shin. The IMUs 34 communicate with one another in order to read the difference in angle between them at any point in the gait cycle.

Additionally, the exosuit 10 may include an air pressure sensor (not shown). In the illustrated embodiment, a control box 38 is coupled to a user's waist (e.g., on a waist belt 40). The control box 38 may house the air pressure sensor and a pump (not shown), which together make up a pneumatic system. This minimizes weight on the foot and decreases circuitry complexity. Conduit 42 (e.g., plastic tubing) provides fluid communication between the pump and the actuators 18, 22.

Together, the sensors 30, 34 form a control system designed to monitor where the user is at every point of the gait cycle. The IMUs 34 monitor the angle between the foot and the leg to ensure that the foot is not extended. The rear force sensors 30 inform the system of heel strike and stance phase in combination with the front force sensors 30 which detect stance phase and push off. These sensor values are communicated to the air pressure sensor and the pump in order to adjust an internal pressure in the side actuators 18 and the front actuator array 22. The pump provides a fluid (e.g., air) to the actuators 18, 22 through the conduit 42, and the air pressure sensor monitors the internal pressure of each actuator 18, 22. This helps to ensure that each actuator 18, 22 is properly inflated at each phase of the gait cycle (e.g., heel strike, stance phase, push off, and swing phase). For example, the side actuators 18 inflate and the front actuator array 22 deflates at the beginning of heel strike. During stance phase, the side actuators 18 maintain their inflated state. At push off, the side actuators 18 are exhausted to a deflated state and the front actuator array 22 inflates. Finally, during the swing phase, front actuator array 22 maintains its inflated state.

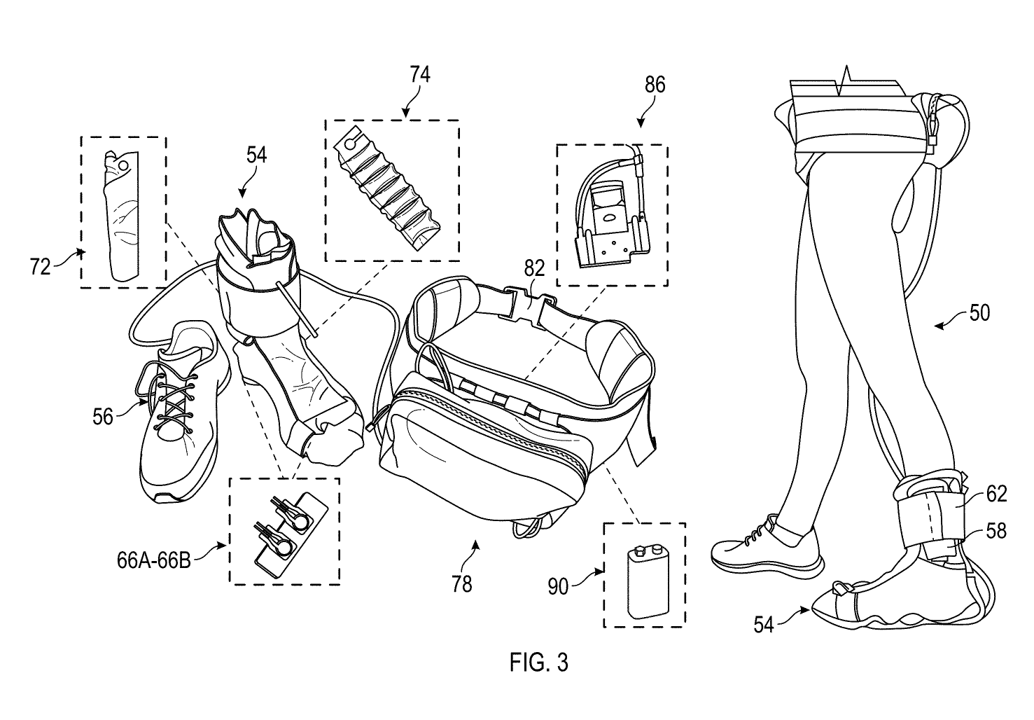

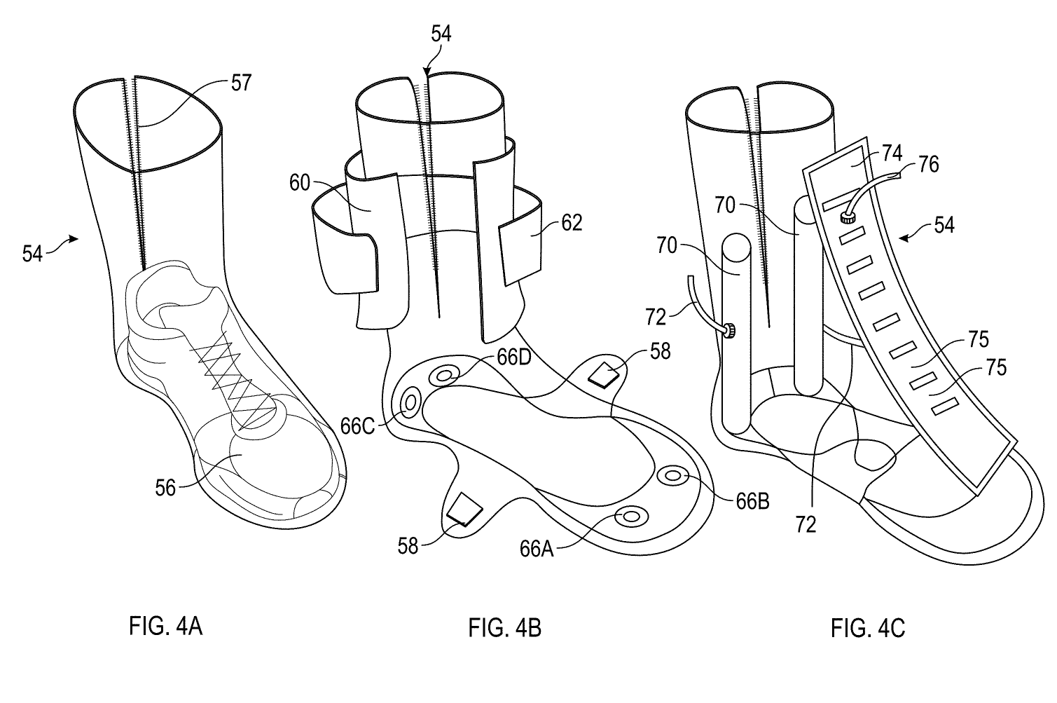

As shown in FIGS. 3 and 4A-C, another embodiment of an exosuit 50 (e.g., a soft Ankle and Foot Orthoses (AFO) exosuit) includes a brace or sock 54 worn on the foot of the user. In the illustrated embodiment, the sock 54 is worn over top of a user's shoe 56. The sock 54 is made from an extensible fabric sleeve (e.g., an elastic spandex material), which may stretch to assist a user in putting on or taking off the sock 54. The sock 54 also includes a zipper 57 (see e.g., FIG. 4A) that permits an opening of the sock 54 to expand. This allows a user to fit the sock 54 over the foot and shoe 56, but also secure the sock 54 against the user's leg. The sock 54 also includes straps 58 (e.g., Velcro straps, see FIG. 4B) coupled to a top of the sock 54 (i.e., proximate the laces). The straps 58 releasably wrap around the shoe 56 in order to adjust the tightness of the sock 54 against the user's shoe 54.

Pads 60 are coupled to the sock 54, and assist in limiting slippage between the sock 54 and the skin of the user. The pads 60 assists in maintaining the shape of the sock 54. In the illustrated embodiment, the exosuit 50 includes two pads 60, one positioned proximate each ankle. A strap 62 (e.g., a Velcro strap) wraps around the sock 54 and couples the pads 60 against the sock 54 and the user's leg. In other embodiments, the pads 60 may be secured to the sock 54 in other ways, or the exosuit 50 may not include pads.

A portion of the sock 54 may also be made from an inextensible fabric (e.g., nylon). In the illustrated embodiment, the base of the sock 54 (i.e., proximate the sole of the shoe 56), is made from the inextensible fabric and provides a point to couple sensors 66A-66D to the sock 54. The inextensible fabric also reduces slippage between the sock 54, shoe 56, and sensors 66A-66D.

The sensors 66A-66D are coupled to the sock 54 and positioned proximate the sole of the shoe 56. In the illustrated embodiment, the sensors 66A-66D are force sensors (e.g., FSR sensors) and detect the placement of the foot by reading ground reaction forces (GRF). Two FSR sensors 66A, 66B are disposed below the ball of the foot, and two FSR sensors 66C, 66D are disposed below the heel. The FSR sensors 66A-66D allows the exosuit 50 to determine where the user's weight is distributed.

The exosuit 50 includes variable stiffness or side actuators 70, which are coupled to the sock 54 on either side of the ankle. The side actuators 70 give support to the ankle to prevent ankle buckling during the heel strike phase of the gait cycle. The side actuators 70 also assist in ensuring proper proprioception before planting the foot on the ground. The side actuators 70 also work together to orient the ankle during each phase of the gait cycle. A conduit 72 (e.g., a plastic tube) extends from a side of each side actuator 70, and provides a fluid pathway into each respective side actuator 70.

The exosuit 50 also includes a dorsiflexion actuator or actuator array 74 that is coupled to the front of the sock 54 (i.e., proximate the laces of the shoe 56) and between the side actuators 70. The actuator array 74 may be a single fluid bladder, or may be a series of interconnect fluid bladders 75. The actuator array 74 creates a bending moment when inflated, and assists a user in the motion of lifting his or her toes toward the body. A conduit 76 (e.g., a plastic tube) extends from the actuator array 74, and provides a fluid pathway into the actuator array 74.

In the illustrated embodiment, each actuator 70, 74 is made from 70 D (Denier) Ripstop Nylon coated in TPU, although other material may be used. Two layers of fabric are stacked on top of one another, with the TPU sides facing one another. The layers are then coupled together (e.g., using a heated soldering iron). The actuators 70, 74 may be substantially similar to the respective actuators 18, 22 of the exosuit 10.

As shown in FIG. 3, in some embodiments a compartment 78 can be removably coupled to a user's waist (e.g., via a waist belt and buckle 82). A portable pump 86 may be housed within the compartment 78, and may be in fluid communication with the actuators 70, 74 via the conduits 72, 76. Together, this makes up a pneumatic system. The compartment also houses a pressure sensor (not shown) and a battery 90. The pressure sensors are in communication with the FSR sensors 66A-66D and the pump 86 in order to supply the appropriate fluid pressure to the actuators 70, 74 based on the position of the foot. In the illustrated embodiment, the side actuators 70 are synchronized (i.e., inflated and deflated together) by the pump 86. The battery 90 supplies power to the pump 86, as well as the sensors 66A-66D. In the illustrated embodiment, the battery 90 is an 11.1 V LiPo battery pack, although other types of batteries may be used. The pump 86 and battery 90 are housed in the compartment 78 (e.g., off of the sock 54) in order to reduce the excess weight on a user's foot.

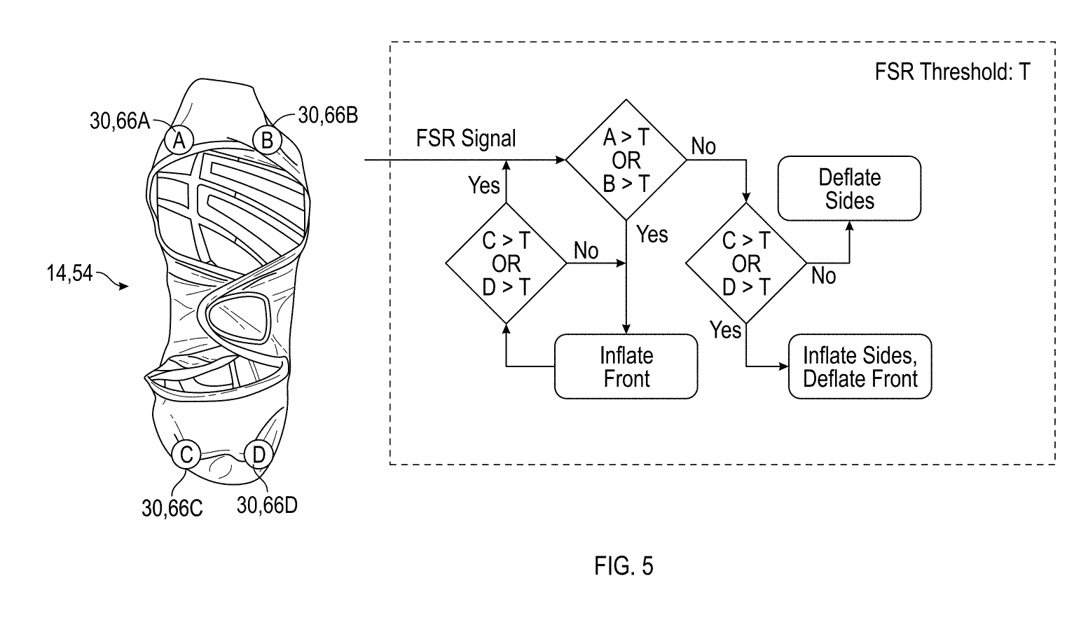

The timing of each section of actuator activation may be determined by thresholds within the control system on each of the sensors (e.g., 30, 66A-66D). These thresholds may be tuned to provide accurate timing for the different periods in the gait cycle for the each user. These thresholds may be tuned differently depending on the weight, foot size, and foot shape, of a different user. As shown in FIG. 5, a first pair of sensors (e.g., 66A, 66B) may be embedded in the anterior side of the sock 54 and a second pair of sensors (e.g., 66C, 66D) may be affixed to the posterior side of the sock 54. This allows the system to identify where the user is at within the gait cycle by monitoring the GRF. In combination, these two sensor pairs (e.g., 66A, 66B and 66C, 66D) read when the user is in the pre-swing or push off phase and when the user is in the heel strike phase of their gait. The system may use a simple bang control logic to monitor a threshold TH for the GRF of the user. When the threshold TH (e.g., more than 10% of the user's normalized weight), is passed the pump is activated and the valves leading to the corresponding actuators are toggled. For example, when the threshold TH is exceeded in either sensor of the first pair of sensors (e.g., 66A, 66B), the actuator array 22, 74 may be inflated until the threshold TH is exceeded by either sensor of the second pair of sensors (e.g., 66C, 66D). This corresponds to the interval between when a user pushes off with the toes to when the heel returns to the ground. Alternatively, if either sensor of the second pair of sensors (e.g., 66C, 66D) exceeds the threshold TH and either sensor of the first pair of sensors (e.g., 66A, 66B) has not, the side actuators 18, 70 may inflate until either sensor of the second pair of sensors (e.g., 66C, 66D) no longer exceeds the threshold TH. In the illustrated embodiment, two fluidic pressure sensors are located in the controls pouch 38, 78 and monitor the activation and deactivation of the two actuators (18, 70 and 22, 74) in the soft AFO exosuit 10 throughout walking. This ensures that the actuators (18, 70 and 22, 74) are inflating properly and the pneumatic system is triggering at the appropriate times.

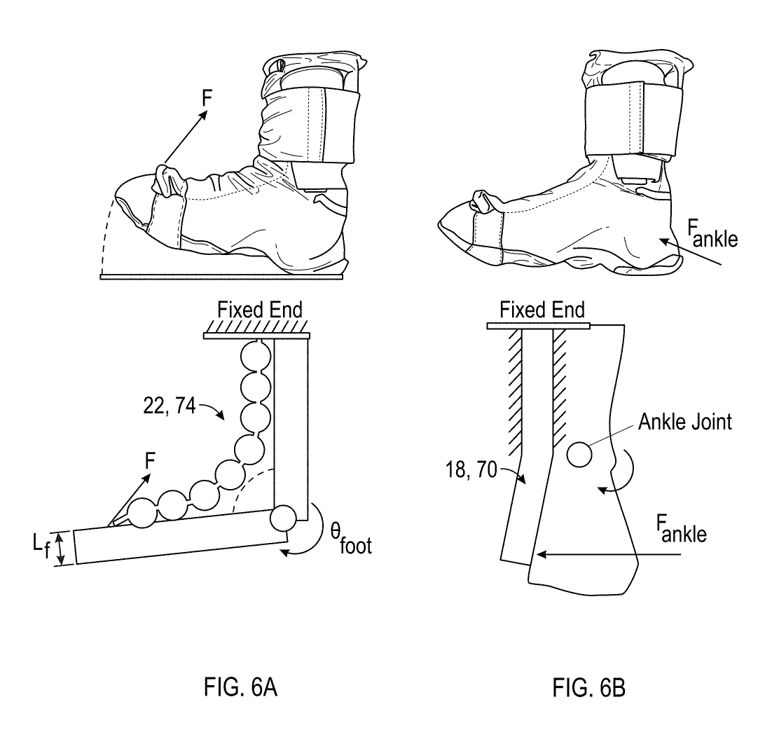

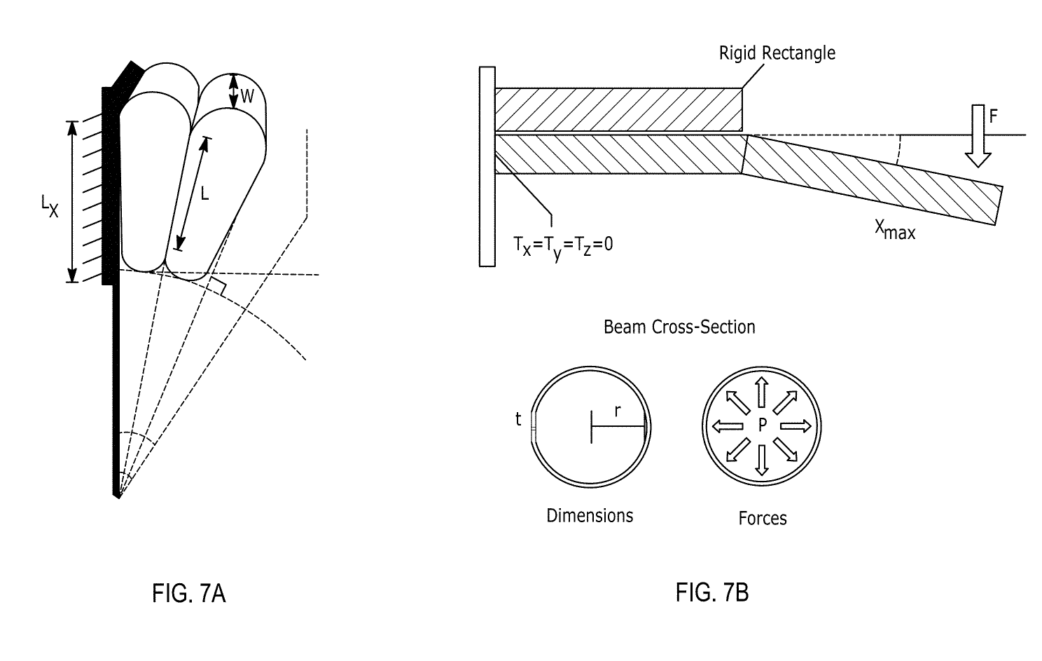

As shown in FIGS. 6A, 6B, and 7A, the actuator array 22, 74 was modeled assuming that each bladder 24, 75 in the actuator array 22, 74 is a deformed sphere, constrained along one edge to provide a bending motion when inflated. The actuator array 22, 74 of the exosuit 10 provides the bending motion to provide swing phase assist. It is assumed that all torque will come from the end level arm and that the forces between bladders 24, 75 rely on the pressure given to the actuator array 22, 74. In the illustrated embodiment, the bladders 24, 75 remain under 5 cm in height to avoid adding excess bulk to the exosuit 10, 50. A width of the actuator array 22, 74 may be approximately the width of the average adult male foot. The final torque T output of the system is modeled below as,

T=PLL_{F}w (1)

where P is the input pressure, L is the length of the contact between two actuators, L_{f} is the length of the lever arm, and w is the width of the contact area between each bladder 24, 75.

As shown in FIG. 7B, the side actuators 18, 70 were modeled using principals of beam theory, and assuming the actuators 18, 70 will behave similar to that of a cantilever beam. Because the side actuators 18, 70 are providing stiffness rather than dynamic assistance, the boundary conditions for the beam are fixed such that the beam is fixed along one side where it is in constant contact with the ankle. The side actuators 18, 70 of the exosuit 10 provide ankle support and proprioception while a user is standing. The other half of the beam is modeled with an applied point load from the base of the calcaneus as the ankle begins to supinate. The deflation of the each side actuator 18, 70 is minimized using the following equation for deflection.

x_{deflection}=FL³/3EI (2)

where F is the force of the ankle acting against the actuator, L is the length of the side actuator, E is the Young's Modulus of 40 D Ripstop Nylon, and I is,

I=¼πr⁴ (3)

where r is the radius of the actuator 18, 70. The only variable value is the radius, and so increasing the radius of the actuator 18, 70 will decrease the overall deflection when a force F is applied.

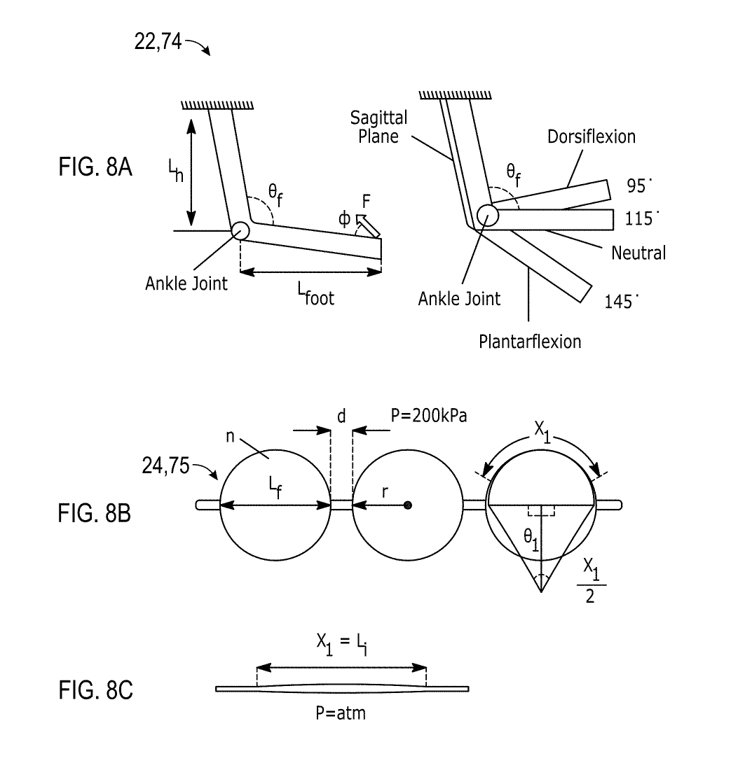

As shown in FIGS. 8A-8C, the actuator array 22, 74 has been modeled to determine the needed amount of contraction for the actuator array 22, 74. The method for identifying the amount of contraction needed to perform dorsiflexion is determined using the law of cosines

Lₐₚₚᵣₒₓ(θ_{f})=\sqrt{Lₕ²+L_{f}²−2LₕL_{f} cos θ_{f}} (4)

where θ_{foot} is there current ankle angle and ranges from 95°≤θ_{f}≤145° (see e.g., FIG. 8A). The angle θ_{foot} at which the ankle is assumed to be in the natural, relaxed position is at 115°, allowing for 20° dorsiflexion and 30° plantation. Lₕ is the height at which the actuator array 22, 74 is mounted on the shin from the joint, and L_{foot} is length between the joint and the metatarsal at which the actuator array 22, 74 is affixed for applied force. The initial length is assumed to allow for the full range of plantarflexion, while the final length assists in the full range of dorsiflexion. The tension force is assumed to be linearly proportional to the input pressure for the purposes of the following force mode

F=L_{f} sin φ (5)

where, φ is the angle between the F vector and its vertical component. The angle φ is obtained assuming the foot has an upward slope of 25° in the neutral position, which is subtracted off the right angle produced between the ground and the vertical force vector. The resulting maximum force to achieve the desired contraction percentage for the actuator is estimated as

$$\begin{matrix}ɛ = \frac{{L}_{i} - {L}_{f}}{{L}_{i}} & ( 6 ) \\ L ( {X}_{1} ) = ( n - 1 ) d + {nx}_{1} + 2 d & ( 7 )\end{matrix}$$

where Lᵢ is the initial length of the actuator array 22, 74 prior to inflation (see e.g., FIG. 8C). L_{f} is the final length after contraction (see e.g., FIG. 8B). The distance between each bladder 24, 75 is denoted as d, n is the number of bladders 24, 75, and x₁ is the initial height of the deflated bladder 24, 75. In order to predict the final length of the actuator array 24, 75, the contraction is estimated as follows:

$$\begin{matrix}{L}_{f} ( {θ}_{1} ) = \frac{\vert {X}_{1} \vert \sqrt{- 2 ( cos 2 {θ}_{1} ) - 1}}{2} & ( 8 ) \\ {sin}^{- 1} ( \frac{2}{π} ) \leq {θ}_{1} \leq {90}^{∘} & ( 9 )\end{matrix}$$

where, θ₁ is the half angle between the center line of the unit and the edge (see e.g., FIG. 8B). The values of θ₁ ranges from where the bounds are set at the points at which the bladder 24, 75 forms a perfect circle, or becomes perfectly flat. This model bases the final contraction percentage on the input constants d and x₁. Using the specific dimensions of the actuator array 22, 74 of d=0.02 cm and x1=2.5 cm the final contraction ratio is estimated at 36.5% of the initial length, assuming the bladders 24, 75 can inflate to perfect circles. This value is comparable to the values achieved experimentally (e.g., through finite element analysis (FEA) optimization).

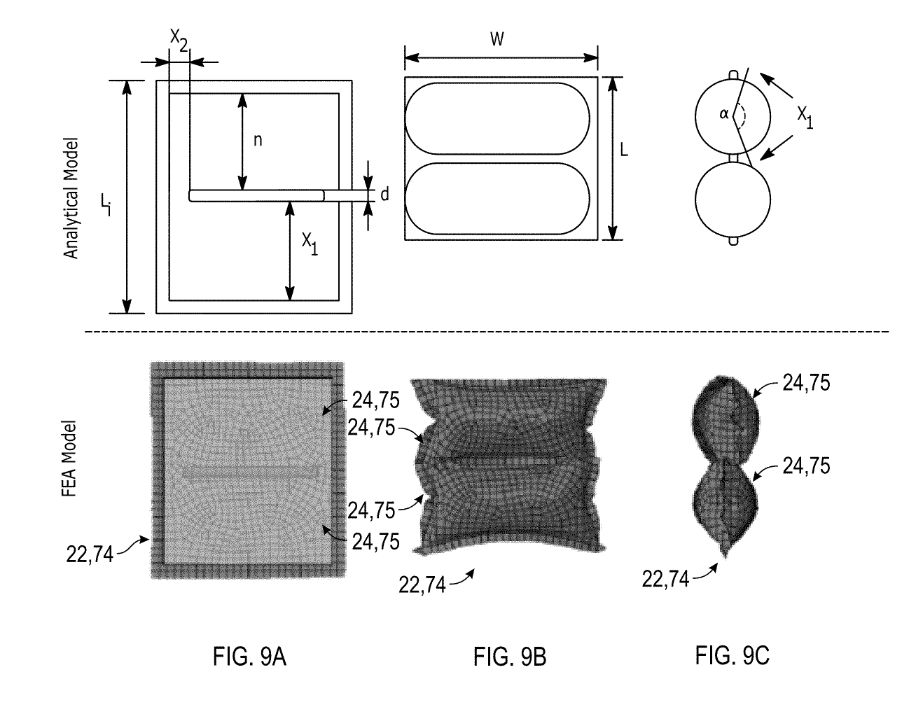

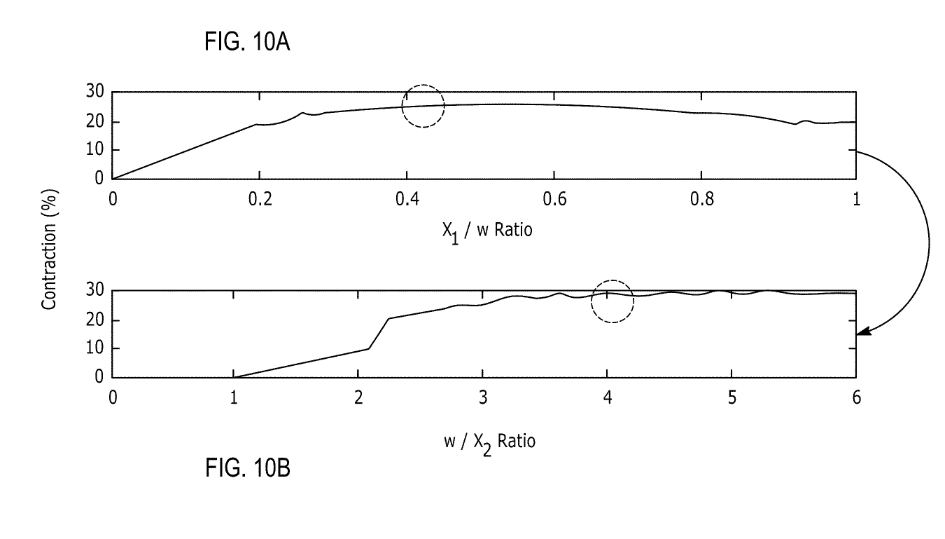

As shown in FIGS. 9A-9C and 10A-10B, the actuator array 22, 74 may be characterized using FEA to model the behavior of the soft materials. In the illustrated embodiment, the models were created using two overlaid 2D homogeneous shell bodies in the rectangular shape of the actuator array 22, 74. The seams were created by implementing tie constraints as denoted by the shaded regions in FIGS. 9A-9C, and the actuator array 22, 74 was fixed along the top seam at Tx,Ty,Tz,Rx,Ry,Rz=0 to prevent movement in the top section during inflation. A uniform pressure force was applied to the internal faces of the two shells to behave as a pneumatic pressure source. The material properties are represented through an elastic behavior, with a Young's modulus of 224 MPa, identified in former studies with TPU coated nylon fabrics. During inflation, the thin shells moved and crumpled as the TPU coated nylon fabric does in reality, and was observed to mimic the behavior of the inflatable fabrics selected.

The dimensions of the actuator array 22, 74 were optimized to maximize contraction percentage while minimizing the internal volume of the actuator array 22, 74. The contraction was observed while varying three parameters individually while holding all other parameters constant the ratio of the height to the width of each unit (h), the gap on either side of the seam connecting each unit (g) and the internal operating pressure (P) of the actuator array 22, 74. It was assumed that these three parameters would have the greatest impact on the overall contraction ratio and are varying sequentially as specified.

The parameter h was evaluated, with the height/width ratio of each unit with all other parameters held constant. First, h was varied from 0.1:1 to a 1:1 ratio. As shown in FIGS. 10A and 10B, a decline in the contraction percentage was observed. The peak contraction percentage was achieved at a ratio of 0.5:1 height/width ratio. The gap size was evaluated as a ratio of the width given by

g(x)=W/x₂ (10)

where, W is the width of the actuator array 22, 74. The gap/width ratio was evaluated from 1:1 to 6:1, at which point the tolerances achievable given the current manufacturing processes was no longer feasible. FIG. 10B shows the maximum contraction obtained with a gap/width ratio, g, of 5.3/1. However, due to physical limitations such as inflation time of the actuator array 22, 74, this gap size was increased to a ratio of 4:1 to allow for better airflow with the particular width selected.

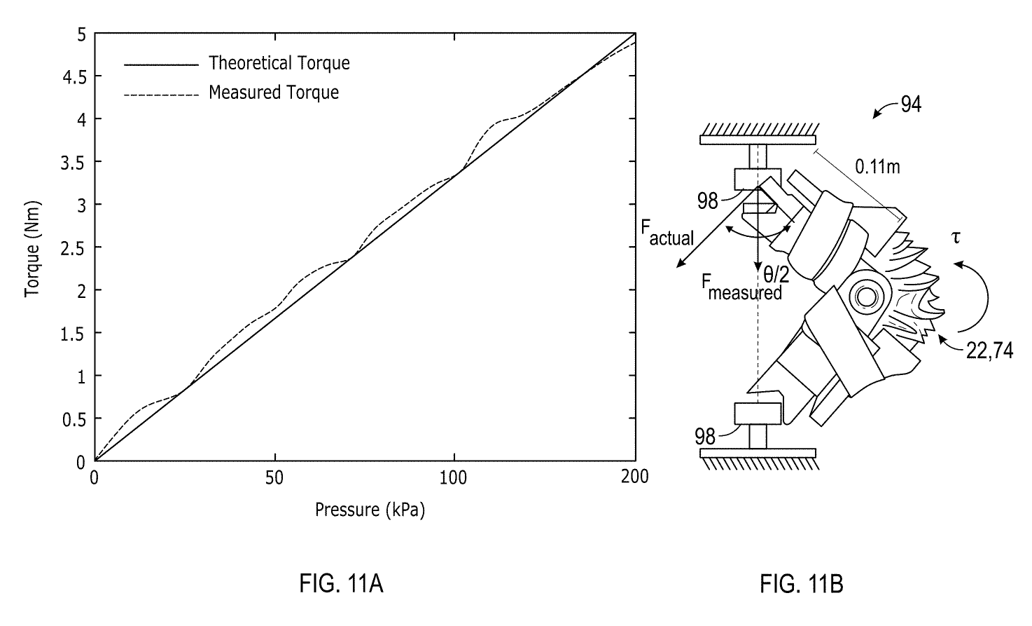

To verify the predictions made in the analytical models, both the actuator array 22, 74 and side actuators 18, 70 were tested for torque and stiffness, respectively. As shown in FIGS. 11A and 11B, the actuator array 22, 74 was affixed to an analog joint, which was connected to a universal tensile strength machine 94 (UTM). In the illustrated embodiment, the pressure was incrementally increased to 150 kPa during 5 separate trials. The illustrated actuator array 22, 74 had a maximum torque value of 4 Nm of Torque. This torque value can be achieved at roughly 110 kPa, and the pressure vs. Torque output approximately corresponds to the theoretical data with an error of less than 5%.

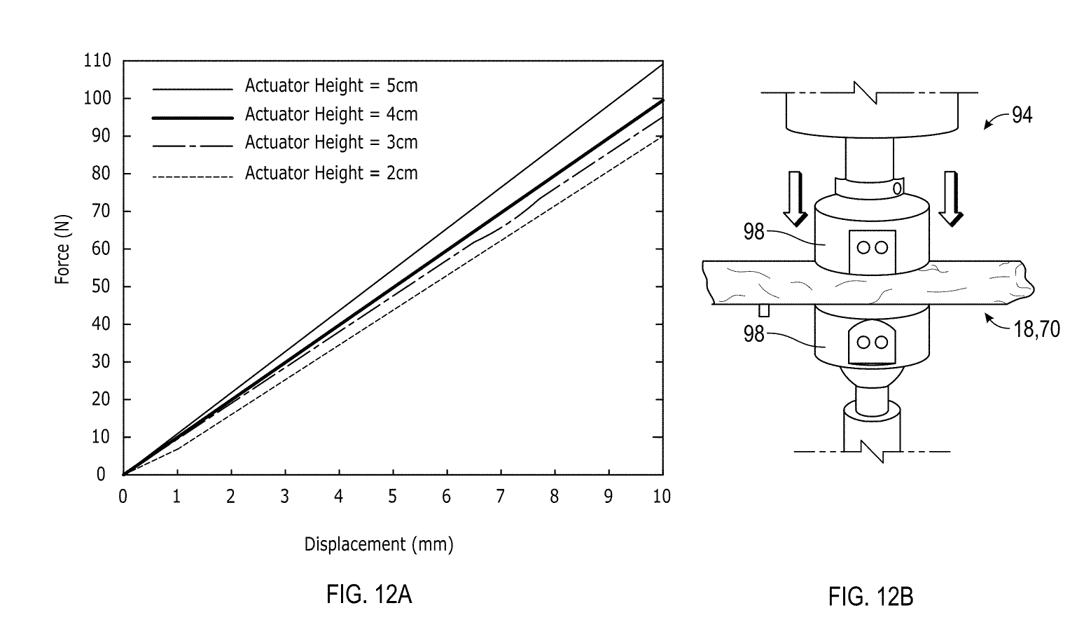

As shown in FIGS. 12A and 12B, four different sized side actuators 18, 70 were tested (e.g., 2 cm, 3 cm, 4 cm, 5 cm) to determine how the radius of the side actuators 18, 70 affects the stiffness of the side actuators 18, 70. The values used in the test were chosen based on various manufacturing and design characteristics. For example, the side actuators 18, 70 had to be large enough (e.g., larger than 1 cm)—in order to allow a tube to fit in a side actuator 18, 70. Additionally, side actuators 18, 70 that are too large (e.g., larger than 6 cm) become impractical to wear on the ankle. Each of the side actuators 18, 70 were placed in the UTM 94 resting between two platens 98 at a fixed distance approximately the same as the diameter of the inflated side actuator 18, 70. The side actuator 18, 70 was compressed 1 cm and then released. This was performed for 5 cyclical iterations and the force required to complete the compression was recorded. The results show a linear stiffness, which increased by roughly 5% with each increase in the radius of the side actuator 18, 70.

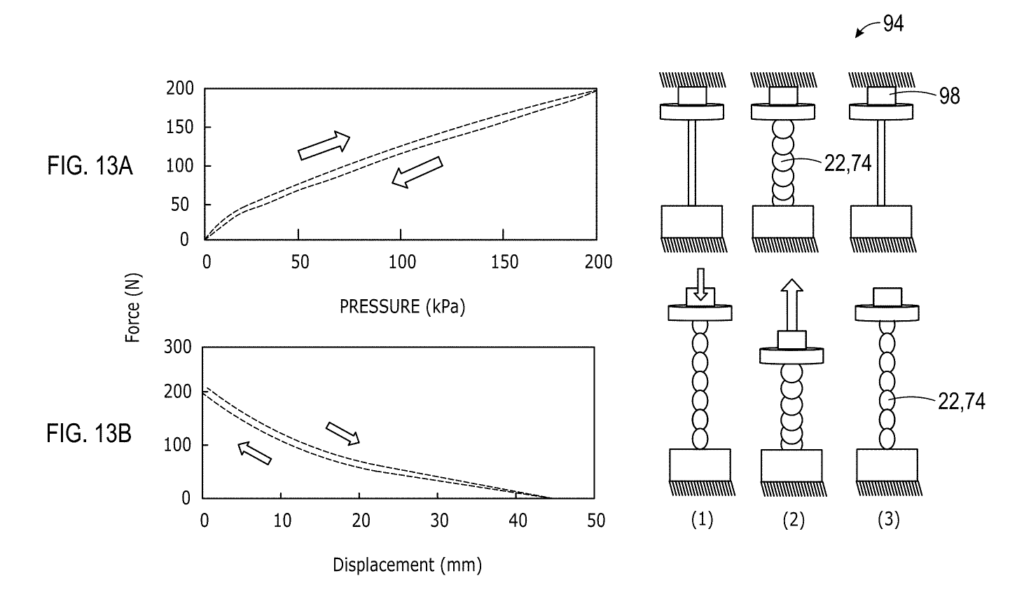

As shown in FIGS. 13A and 13B, a quasistatic isometric test was performed on the actuator array 22, 74 using the UTM 94, where both ends of the actuator array 22, 74 were clamped and held at a fixed position. The pressure was increased in increments of 10 kPa from 0 kPa up to the maximum operating pressure of 200 kPa, and back down to 0 kPa. This was performed cyclically for three trials, with the UTM 94 being reset and recalibrated between each trial. The actuator array 22, 74 was able to generate a maximum of 197.1±2.2N when pressurized up to the maximum operating pressure of 200 kPa while the contraction was 0%.

A constant pressure test was performed with the actuator array 22, 74 on the UTM 94, with pressure held constant at the maximum of 200 kPa throughout the entirety of the test. The actuator array 22, 74 was clamped at both ends, with a load cell 98 affixed to the top end. At the start of the test, the top clamp 98 was released and allowed to move freely downward to measure the contraction of the actuator array 22, 74, as well as the pulling force generated. The clamp X was released from its position, moved downward until the force measured reads ON, then moved back to its original fixed position. This was repeated cyclically for three trials for the actuator array 22, 74. A total displacement of 43.1 mm±0.5 mm was observed, or a contraction of 32.3%, compared to the 36.5% predicted assuming ideal contraction of each unit to form a perfect circle.

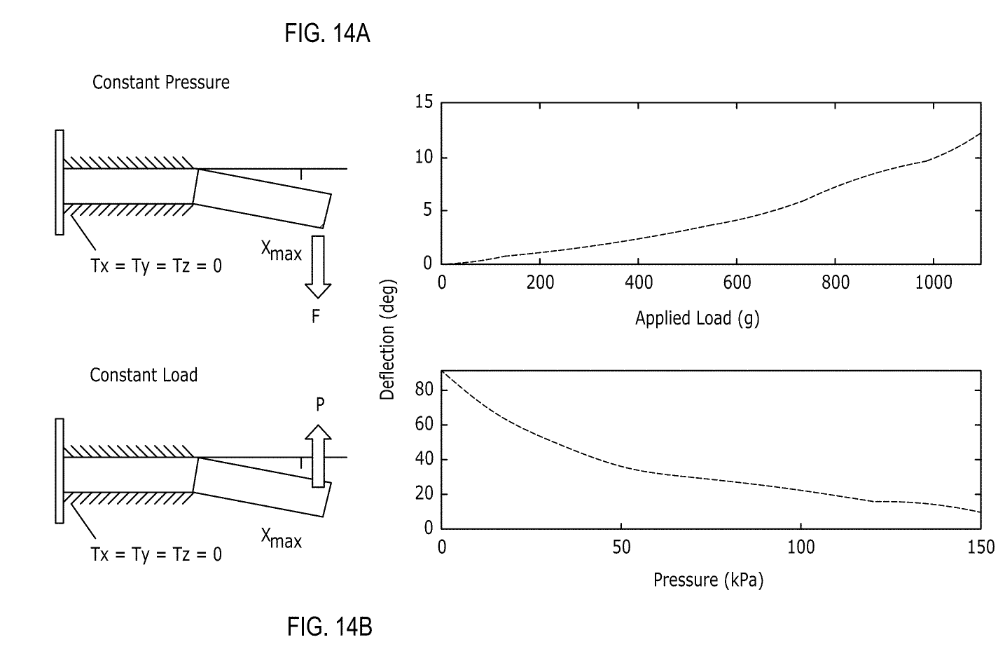

As shown in FIGS. 14A and 14B, the side actuators 18, 70 were evaluated based on the ability to resist the lateral and medial movement of the ankle, which can lead to excessive supination or pronation of the ankle and cause buckling. A single side actuator 18, 70 was placed in a custom vice with half of the side actuator 18, 70 length fixed in place and the other free-floating as a cantilever beam. This mimicked the placement of the side actuators 18, 70 on the ankle, fixed at one end by the straps 16, 62 of the exosuit 10, and the other free to bend and move against the calcaneus of the ankle.

The side actuator 18, 70 was evaluated under constant pressure of 150 kPa, with increasing load applied at the end of the cantilevered end. The angle of deflection of the side actuator 18, 70 was measured each time load is added, and this was repeated for three iterations. It was found that the side actuator 18, 70 can stay under 10±0.17° deflection with a load of 1 Kg applied at the tip. The side actuator 18, 70 was also assessed with constant load with varying pressures. The system was pre-loaded with 1 kg, the equivalent of 1.2 Nm of torque with the actuator fully deflated. This was roughly 12% of the maximum ankle torque during rapid buckling The side actuator 18, 70 was then inflated in increments of 10 kPa up to 150 kPa with the constant load still in effect. A deflection from a fully bend actuator at 90° to 12.0±0.46° state of deflection was observed.

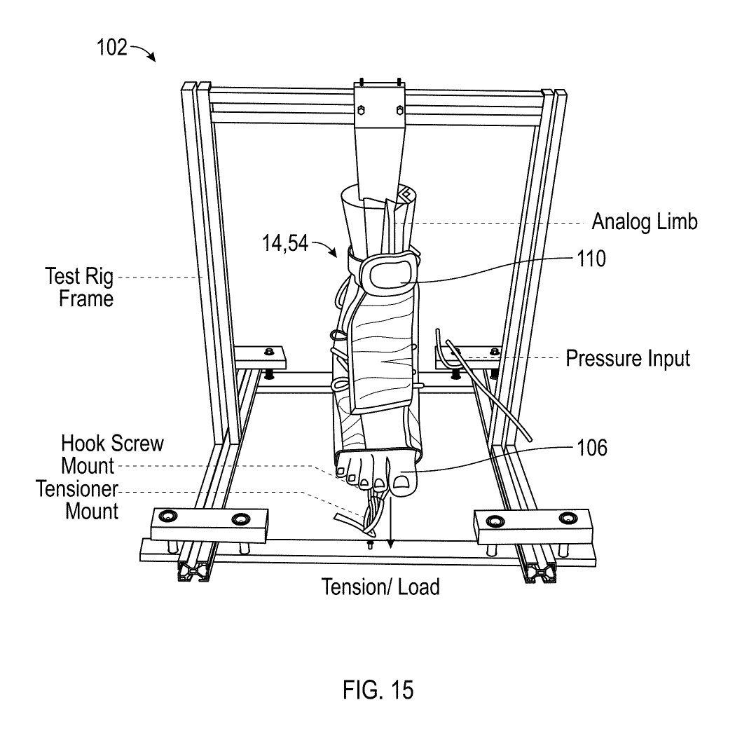

As shown in FIG. 15, a test platform 102 was constructed in order to test how the brace 14, 54 would affect the end user's range of motion. In the illustrated embodiment, the test platform 102 was constructed using aluminum extrusions attached with rigid brackets. A test foot 106 was 3D printed and attached to a universal joint 110 to act as a non-flexible stand in to demonstrate the full range of motion of the actuators 18, 22, 70, 74. A baseline test was performed on the test foot 106 without the brace 14, 54 in order to find the range of motion of the test foot 106. Once the baseline was captured (e.g., using a motion capture system—not shown), a subsequent testing was performed with the brace 14, 54 on the test foot 106 to demonstrate the range of motion of the test foot 106 with the brace 14, 54 in place.

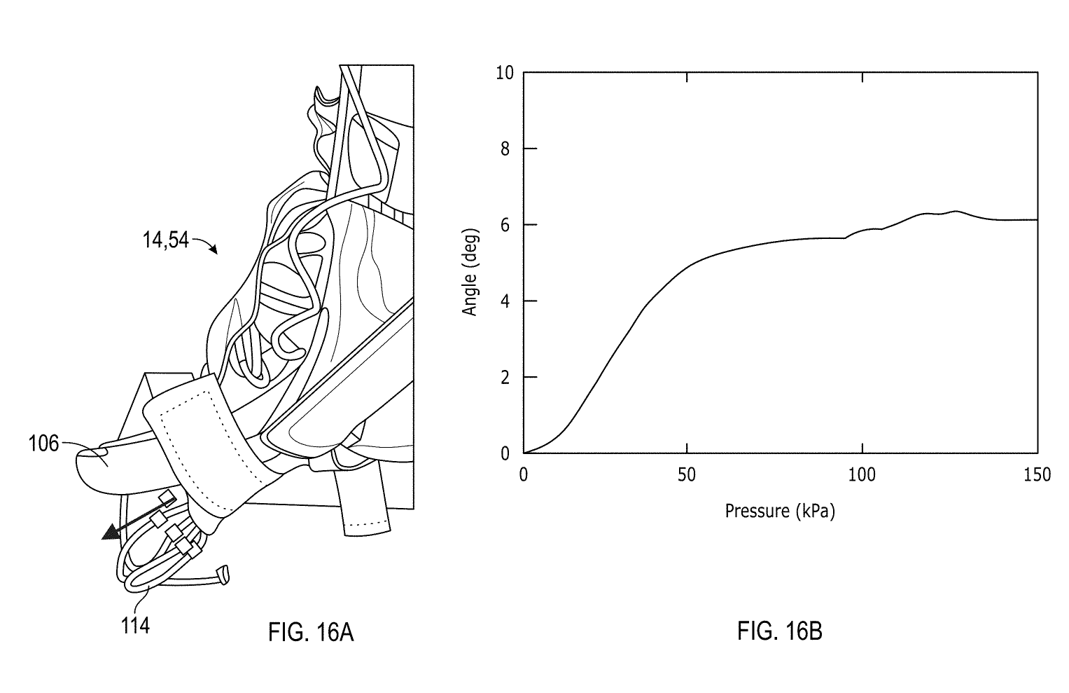

As shown in FIGS. 16A and 16B, the test foot 106 was attached to resistive cabling 114 after the range of motion tests were performed. An additional test was performed to show that the actuators 18, 22, 70, 74 of the exosuit 10, 50 could provide more than the required 4 Nm of torque to the system allowing for the support of dorsiflexion.

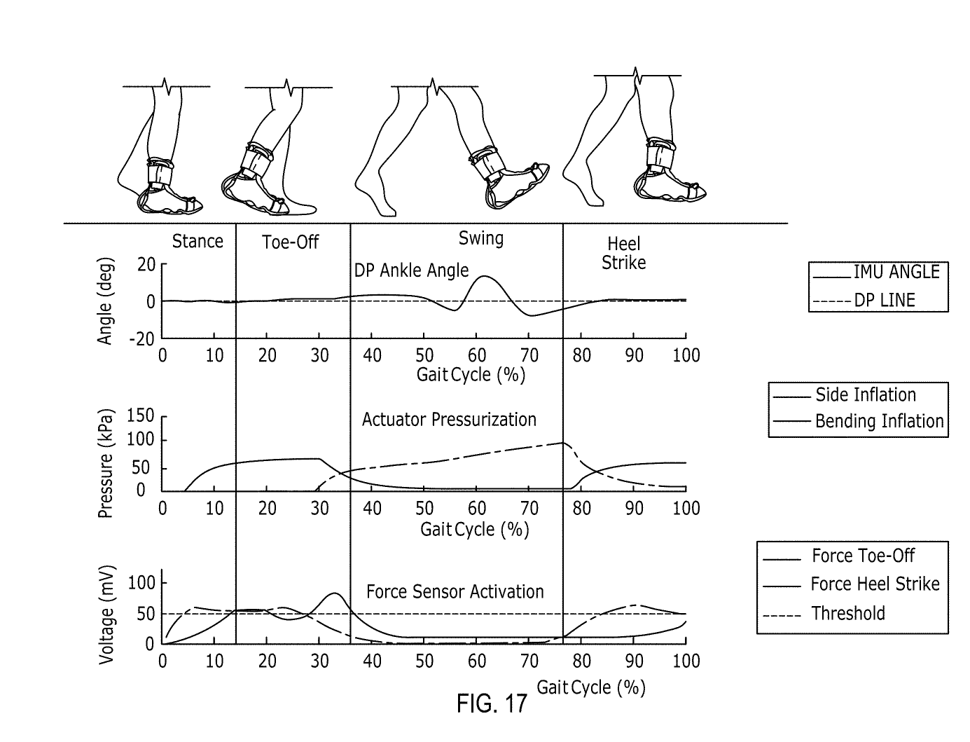

The exosuit 10, 50 was worn by a user in order to confirm that the exosuit 10, 50 worked in practice. The tester walked the length of the test area at a slow pace emulating a patient going through physical therapy that might not be able to walk quickly. As the tester walked, the data from all of the sensors (e.g., the FSR 30, the IMU 34, and the air pressure sensors) was logged. This data can be seen in FIG. 17.

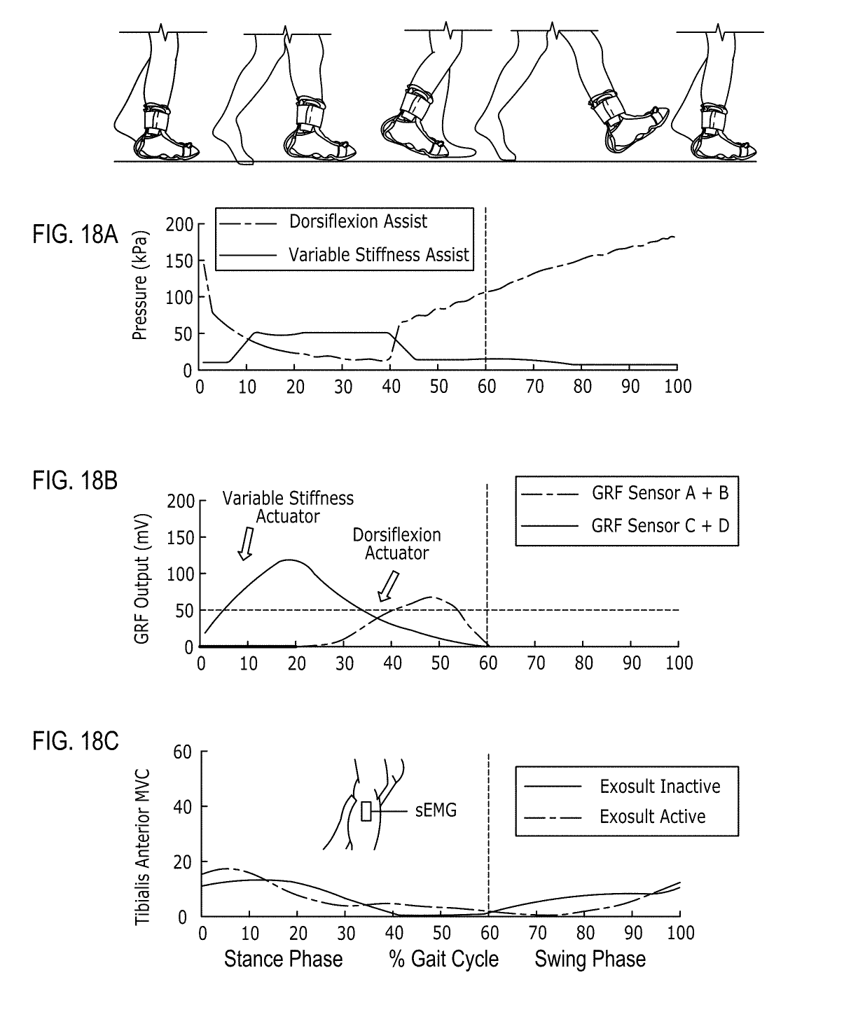

As shown in FIGS. 18A-18C, the exosuit 10 was also tested to determine if a user experiences less muscle activity in the tibialis anterior during the swing phase of the gait cycle while wearing the exosuit 10, 50. The test was performed with and without the exosuit 10, 50 active. An average across trials of 21.3% decrease in muscle activity was observed during dorsiflexion when isolating the swing phase of the gait from 60% to 100%. These results indicate that the exosuit 10, 50 was able to reduce muscle effort in the main muscle related to dorsiflexion during the swing phase in a healthy participant.

The embodiment(s) described above and illustrated in the figures are presented by way of example only and are not intended as a limitation upon the concepts and principles of the present disclosure. As such, it will be appreciated that variations and modifications to the elements and their configuration and/or arrangement exist within the spirit and scope of one or more independent aspects as described.

What is claimed is:

1. A soft robotic ankle-foot orthosis exosuit comprising:
a brace configured to be worn on a user's foot;
a first soft actuator coupled to the brace and configured to be positioned proximate a top of the user's foot;
a second soft actuator coupled to the brace and configured to be positioned proximate a side of the user's foot; and
a pneumatic system configured to change an internal pressure of the first soft actuator and the second soft actuator.

2. The soft robotic ankle-foot orthosis exosuit of claim 1, further comprising a third soft actuator coupled to the brace and configured to be positioned proximate an opposite side of the user's foot.

3. The soft robotic ankle-foot orthosis exosuit of claim 2, wherein second soft actuator and the third soft actuator are uniformly inflated and deflated by the pneumatic system.

4. The soft robotic ankle-foot orthosis exosuit of claim 1, further comprising force sensitive resistive sensors coupled to a bottom of the brace, the force sensitive resistive sensors configured to measure a ground reaction force of the brace, and the pneumatic system configured to use the measured ground reaction force to change the internal pressure in the first soft actuator and the second soft actuator.

5. The soft robotic ankle-foot orthosis exosuit of claim 1, wherein the brace is configured to be worn over a user's shoe.

6. The soft robotic ankle-foot orthosis exosuit of claim 1, wherein the first soft actuator is an actuator array having a plurality of interconnected fluid bladders.

7. The soft robotic ankle-foot orthosis exosuit of claim 1, further comprising a compartment configured to be worn on a user's waist, the compartment supporting the pneumatic system.

8. The soft robotic ankle-foot orthosis exosuit of claim 1, wherein the second soft actuator is configured to extend along a user's ankle and branches off to extend toward a metatarsal and a calcaneus of the user's foot.

9. A soft robotic ankle-foot orthosis exosuit comprising:
a brace configured to be worn on a user's foot;
sensors coupled to the brace;
a first soft actuator coupled to the brace and configured to provide dorsiflexion assistance to the user's foot;
a second soft actuator coupled to the brace and configured to limit inversion or eversion of the user's foot; and
a pump in fluid communication with the first soft actuator and the second soft actuator, the pump configured to adjust an internal pressure of the first soft actuator and an internal pressure of the second soft actuator based on measurements from the sensors.

10. The soft robotic ankle-foot orthosis exosuit of claim 9, wherein the first soft actuator and the second soft actuator are made from a thermoplastic polyurethane material.

11. The soft robotic ankle-foot orthosis exosuit of claim 9, wherein the first soft actuator is an actuator array having a plurality of interconnected fluid bladders.

12. The soft robotic ankle-foot orthosis exosuit of claim 9, further comprising a pad coupled to an outer surface of the brace, the pad configured to assist in limiting slippage of the brace between a leg of the user and the brace.

13. The soft robotic ankle-foot orthosis exosuit of claim 9, wherein the sensors are inertial measurement units, which are configured to measure an angle between the user's foot and a user's leg.

14. The soft robotic ankle-foot orthosis exosuit of claim 9, wherein the sensors are force sensitive resistive sensors, which are configured to measure a ground reaction force from the brace.

15. The soft robotic ankle-foot orthosis exosuit of claim 9, further comprising a compartment configured to be worn on a user's waist, the compartment supporting the pump.

16. A soft robotic ankle-foot orthosis exosuit comprising:
a brace configured to be worn on a user's foot;
an actuator array coupled to the brace proximate a top of the user's foot, the actuator array including multiple interconnected fluid bladders;
a first side actuator coupled to a first side of the brace;
a second side actuator coupled to a second side of the brace; and
a pneumatic system supported by the user away from the brace, the pneumatic system configured to substantially uniformly change an internal pressure in the interconnected fluid bladders and substantially uniformly change an internal pressure in the first and second side actuators.

17. The soft robotic ankle-foot orthosis exosuit of claim 16, further comprising
a first force sensitive resistive sensor coupled to a bottom of the brace and positioned proximate a front of the brace; and
a second force sensitive resistive sensor coupled to the bottom of the brace and positioned proximate a rear of the brace, the first and second force sensitive resistive sensors configured to measure ground reaction forces from the brace and communicate the measurements to the pneumatic system to adjust the internal pressures.

18. The soft robotic ankle-foot exosuit of claim 17, wherein the pneumatic system further includes
a pump supplying pressure to the actuator array and the first and second side actuators based on the measurements from the first and second force sensitive resistive sensors; and
an air pressure sensor measuring the internal pressure of the actuator array, the first side actuator, and the second side actuator, in order to regulate the pressure supplied by the pump.

19. The soft robotic ankle-foot exosuit of claim 16, wherein the first and second side actuators are configured to be inflated while the actuator array is deflated.

20. The soft robotic ankle-foot exosuit of claim 16, further comprising a pad coupled to an outer surface of the brace, the pad configured to assist in limiting slippage of the brace between a leg of the user and the brace.
